(19) United States
(12) Patent Application Publication
(10) Pub. No.: US 2026/0183132 A1
(43) Pub. Date: Jul. 2, 2026

(54) ELBOW BRACE AND PAD

(71) Applicant: Sporlastic GmbH & Co. KG, Nürtingen (DE)

(72) Inventors: Lukas LAITZSCH, Berlin (DE); Christian HORST, Kirchheim U. Teck (DE); Stephan BLANC, Winterthur (CH); Timo SCHMELTZPFENNING, Tübingen (DE)

(21) Appl. No.: 19/426,570

(22) Filed: Dec. 19, 2025

(30) Foreign Application Priority Data
Dec. 23, 2024 (DE) 102024139647.4

(51) Int. Cl. A61F 5/01; A61F 5/32

(52) U.S. Cl. CPC A61F 5/0118; A61F 5/32

(57) ABSTRACT

The invention relates to an elbow brace (10) for relieving radial and/or ulnar forearm muscles (12, 14) of a patient, comprising at least one shell element (16) for medial and/or lateral arrangement on the forearm of the patient; at least one strap element (18) for fastening the at least one shell element (16) to the forearm of the patient; at least one pad (42) arranged in a defined manner on the at least one shell element (16) with at least one rib (54) extending along a rib axis (52), wherein the pad (42) can be arranged in a defined first configuration in which the pad (42) can be arranged on the radial forearm muscles (12), and a defined second configuration in which the pad (42) can be arranged on the ulnar forearm muscles (14).

The invention relates to an elbow brace, also called an epicondylitis brace, with a releasable or non-releasable pad, and to a pad for arrangement on the elbow brace.

EP2977031 describes an elbow brace with two pads, wherein the pads are arranged on a curved molded part by means of a hook-and-loop fastener.

The invention is based on the object of providing an elbow brace which addresses the disadvantages of the prior art, in particular improving the pressure effect on the forearm muscles of the patient and exhibiting high flexibility in the treatment of a radial and ulnar forearm muscles of the patient.

The object addressed by the invention is achieved by an elbow brace having the features of claim 1. The invention relates to an elbow brace for relieving radial and/or ulnar forearm muscles of a patient, comprising: at least one shell element for medial and/or lateral arrangement on the forearm of the patient; at least one strap element for fastening the at least one shell element to the forearm of the patient; at least one pad arranged on the at least one shell element with at least one rib extending along a rib axis. The pad can be set up in a defined first configuration and a defined second configuration. In the defined first configuration, the pad can be used to treat the radial forearm muscles (“tennis elbow”). In the defined second configuration, the pad can be used to treat the ulnar forearm muscles (“golfer's elbow”).

For the purposes of the invention, a “defined configuration” means that the pad, in the wearing state, is arranged in a repeatable spatial position relative to the forearm muscles. If the change in the configuration of the pad is achieved by changing the position and/or orientation of the pad on the shell element, the “defined configuration” means that the pad is arranged in a repeatable spatial position relative to the shell element. If the change in the configuration of the pad is achieved by changing the position and/or orientation of the shell element, the “defined configuration” means that the pad is arranged in a repeatable spatial position relative to a forearm receptacle and/or to the forearm muscles.

By allowing the pad to be set up in two configurations, the elbow brace can be used flexibly to treat the radial and ulnar forearm muscles, wherein the at least one shell element and the at least one pad can be used. Therefore, for example, a pad can be used to treat both the radial and ulnar forearm muscles. The pad can be placed either dorsally or versally and/or either on the radial or ulnar forearm muscles. Consequently, the pad is particularly flexible in its use. Thus, tennis elbow or golfer's elbow can be treated with the elbow brace.

For the purposes of the invention, a “pad” is understood to be a support body which exerts a massage effect on the adjacent muscles. In this process, the muscle tissue lying beneath the pad is subjected to a shear or tension load, which is particularly variable temporally. For the purposes of the invention, a pad is understood to mean only such support bodies which are fastened to the body of the patient or a part of their body by means of a bandage, in particular a bandage that encircles the body or body part, and in particular are pressed against it. Massage mats or even shoe insoles are not to be understood as a pad in the present sense. These are not fastened to the body or body part of the wearer by means of a bandage, but work via the body weight of the patient. The support body according to the invention, on the other hand, preferably acts in a supporting and massaging manner on the body of the patient via pressure applied by means of the aforementioned bandage. In particular, support bodies in the present sense are attached close to muscles and/or joints.

The pad is preferably at least substantially cross-shaped.

An advantageous aspect of the invention provides that the at least one pad has at least two or three ribs extending parallel to the rib axis. This allows for a particularly effective and targeted massage effect on the forearm muscles.

An advantageous aspect of the invention provides that that at least one shell element and at least one pad are releasably or non-releasably connected to each other. Because the pad is releasable, it can be individually adjusted to the patient. Furthermore, the pad is easily replaceable.

An advantageous aspect of the invention provides that the at least one shell element has at least one recess and/or the at least one pad has at least one fastening portion for form-fitting and releasable connection of the at least one shell element and the at least one pad. Accordingly, the pad can be released from the shell element to change the configuration. Alternatively, the shell element can be rotated to change the configuration of the pad. Preferably, the at least one shell element is designed to be releasable from the at least one strap element.

An advantageous aspect of the invention provides that the at least one pad is asymmetrically shaped perpendicular to the rib axis. This can be achieved, for example, by having the at least one rib be positioned off-center relative to a transverse extension of the at least one rib that runs perpendicular to the rib axis. The transverse extension is defined by the two free ends of the pad, which extend away from the rib perpendicular to the rib axis. This means that targeted pressure can be applied to the forearm muscles, especially at the muscle insertion.

An advantageous aspect of the invention provides that the at least one pad in the first configuration is arranged in a first pad orientation on the at least one shell element, wherein the at least one pad in the second configuration is arranged in a second pad orientation on the at least one shell element, and wherein the first pad orientation and the second pad orientation are inverted relative to each other. Accordingly, by rotating the pad, both the radial and ulnar forearm muscles can be treated. For the purposes of the invention, “inversion” means that the pad is rotated from the first pad orientation to the second pad orientation by at least 90°, in particular by at least 135°, preferably by at least 160°, preferably by at least 170°. Preferably, the orientation of the pad is rotated (inverted) by 180° in both configurations.

An advantageous aspect of the invention provides that the at least one shell element in the first configuration of the at least one pad is arranged in a first shell orientation on the at least one strap element and/or on the forearm of the patient, wherein the at least one shell element in the second configuration of the at least one pad is arranged in a second shell orientation on the at least one strap element and/or on the forearm of the patient, and wherein the first shell orientation and the second shell orientation are inverted relative to each other. Accordingly, by rotating the shell element with the pad, both the radial and ulnar forearm muscles can be treated.

An advantageous aspect of the invention provides that the elbow brace has a first shell element and a second shell element. The first shell element and the second shell element are preferably designed separately and/or identically to each other. The shell elements are preferably connected to each other at two opposite ends via two strap elements, in particular straps, wherein in particular a first strap element serves to determine the distance between the shell elements and/or the second strap element serves to attach the elbow brace to the forearm. In the first configuration, the pad for treating the radial forearm muscles (“tennis elbow”) is preferably arranged or positioned on the first shell element, particularly in the first pad orientation. In the second configuration, the pad for treating the ulnar forearm muscles (“golfer's arm”) is preferably arranged or positioned on the second shell element, particularly in the second pad orientation. Preferably, both shell elements are arranged in a first shell orientation or in a second shell orientation, i.e., in the same orientation. Preferably, the orientation or alignment of the pad is rotated (inverted) by 180° in both configurations.

An advantageous aspect of the invention provides that at least one first recess and at least one second recess are provided in the at least one shell element, and wherein the at least one pad is arranged at the at least one first recess in the first configuration and at the at least one second recess in the second configuration.

An advantageous aspect of the invention provides that the at least one shell element has a honeycomb structure. Preferably, at least one recess is part of the honeycomb structure. Due to the combination of a shaping honeycomb structure, a flexible strap element and a locally acting pad, the forearm muscles of a patient can be specifically massaged, so that tension can be relieved by wearing the elbow brace. Due to the honeycomb structure, a particularly lightweight, specifically stabilizing and adaptable as well as aesthetically pleasing elbow brace is provided. Due to the recess in the honeycomb structure, the releasable pad can be easily fastened to the honeycomb structure. No additional fastening means is required for fastening the pad to the structural honeycomb structure. An elbow brace has the advantage over a textile strap that it does not restrict blood circulation and does not constrict the radial or ulnar nerve by pressure in the cross section.

The elbow brace is preferably adjustable in the brace width or dorsally, in particular by means of the at least one strap element. Accordingly, the elbow brace can be adjusted to the arm circumference and the position and direction of the pressure force caused by the pad can be adjusted. A width-adjustable elbow brace ensures that the pressure increase only acts from the sagittal direction. Pressure forces are reduced in the elbow crease and in the dorsal region of the elbow tip, thus preventing constrictions. Furthermore, the position of the pad can also be determined by means of the width adjustment.

For the purposes of the invention, a “honeycomb structure” is to be understood as a stabilizing structural component which has an irregular honeycomb structure over its entire planar extension. The irregular honeycomb structure preferably has webs of strengthening structural material and the webs delimit irregularly shaped recesses free of structural material. In stabilizing regions, the webs can be made thicker than in adaptable regions. The honeycomb structure is preferably designed to be planar. Planar in this context means an embodiment of the honeycomb structure in which the thickness of the honeycomb structure in the direction of the local surface normal is significantly lower than the dimensioning of the honeycomb structure along its planar extension. However, a planar extension does not mean the extension in a geometric plane; instead, the honeycomb structure can have a three-dimensionally sweeping or curved shape. Typically, the honeycomb structure is formed complementary to the limb or body shape to be supported in an area surrounding a joint.

The honeycomb structure can have at least one stabilization direction lying in the planar extension. Transverse to the stabilization direction, the honeycomb structure is designed with local flexural rigidity (in a region of the honeycomb structure) to have a stabilizing effect. In the applied state of the orthosis, the honeycomb structure is arranged such that the stabilization direction of the honeycomb structure runs along the body part, in particular the forearm, of the patient, so that the patient's body part to be stabilized is stabilized by the honeycomb structure. A bending transverse to the stabilization direction is possible only to a minimal degree. The honeycomb structure is thus designed to be rigid transverse to the stabilization direction, such that it serves to support or stabilize a limb of the orthosis patient (in the applied state). Having stabilizing flexural rigidity is understood in this context to mean a flexural rigidity which can permit a certain bending, as is customary in a rail of an orthosis, but in any case is small enough that the supporting function and stabilization of the limb is performed.

In addition to the stabilization direction, the honeycomb structure can have an adaptation direction lying in the planar extension. The honeycomb structure is designed to be locally bendable transverse to the adaptation direction. In this case, the honeycomb structure is designed to be bendable transversely to the adaptation direction such that it can be brought into planar contact with the contour of a limb of an orthosis patient by bending transversely to the adaptation direction. The bending necessary for this can be achieved manually during the application. For example, the orthosis or its honeycomb structure can be fixed by corresponding fastening straps and at the same time be adapted, by bending, to the local body shape of the wearer. The fastening and adaptation by means of the fastening straps also contribute to the secure seating of the brace. Although the honeycomb structure is bendable transverse to the adaptation direction, it offers in particular a certain resistance to a bending, so that it maintains a tension when applied. The discussed bendings of the honeycomb structure as provided by this invention refer to a bending in the direction of the local surface normal to the extension of the honeycomb structure.

In particular, a specific selection of the placement and dimensioning of individual webs and a variation of the number of webs advantageously achieve a local adjustment of the stiffness of the structural component over its planar extension. As a result, individual regions of the honeycomb structure can be designed to be flexurally rigid and other regions can be designed to be flexible.

Various local stiffnesses and degrees of flexibility are easily realizable. The honeycomb structure has in particular a closed edge. Thus, no webs terminate in the edge in the direction of their longitudinal extension. Rather, the edge is formed by a circumferential web. The recesses that are free of material have in particular a plurality of different shapes, in particular at least 3, in particular 5, in particular 7, different shapes of recesses on a honeycomb structure. In particular, the recesses are formed predominantly or exclusively with rounded corners. In the direction of connection points with further webs, the inserts can thicken in the planar extension of the honeycomb structure. In particular, there are different connection points in a honeycomb structure, in particular connection points in which a web impinges on a web extends in a straight line, connection points in which a plurality of webs impinge on a web which extends in a straight manner and connection points in which a plurality of (in particular 3 to 6, in particular up to 5) webs from different directions intersect. The honeycomb structure can comprise connection points in which 2 straight-extending webs intersect. The different connection points can be present in different combinations in the honeycomb structure.

An advantageous aspect of the invention provides that the honeycomb structure with the at least one recess is designed in such a way that the pad can be connected to the shell element in a first orientation and a second orientation, in particular opposite to the first orientation.

An advantageous aspect of the invention provides that the at least one pad, in the intended wearing state, can be connected to the shell element in such a way that the rib axis runs at least substantially parallel to muscle fibers of the radial and/or ulnar forearm muscles of the patient. Therefore, an optimal depth effect of the micromassage of the forearm muscles can be achieved.

An advantageous aspect of the invention provides that the at least one pad has at least one pad nose extending perpendicular to the rib axis for applying pressure to muscle fibers towards the at least one rib. This is particularly advantageous because the forearm muscles in the region of the pad nose can be acted upon towards the rib, so that the rib can act more effectively on the forearm muscles.

An advantageous aspect of the invention provides that the at least one pad has two pad noses. Therefore, the impact effect of the pad can be further increased. In addition, preferably the at least one rib is arranged perpendicular to the rib axis and off-center to the two pad noses. Preferably, one pad nose is longer than the other pad nose, so that a directed impact on the forearm muscles towards the rib can be increased in one direction.

An advantageous aspect of the invention provides that the at least one recess of the honeycomb structure is designed as an aperture through the at least one shell element.

An advantageous aspect of the invention provides that the elbow brace has two shell elements that are separate from each other and, in particular, identical. This means that constriction of blood circulation and compression of the radial or ulnar nerve can be further reduced.

An advantageous aspect of the invention provides that the at least one shell element has at least one, in particular slot-like, first fastening receptacle for the releasable or non-releasable receiving of the at least one strap element. Therefore, the strap element can be easily attached.

An advantageous aspect of the invention provides that the first fastening receptacle has a primary fastening receptacle and a secondary fastening receptacle, wherein the primary fastening receptacle is arranged in a central region and/or the secondary fastening receptacle is arranged in an edge region of the at least one shell element. The end of the strap element, which is arranged in the primary fastening receptacle, i.e., in the central region, is preferably guided along the outside of the shell element. This ensures a targeted deflection of the strap element. This preferably can be used for particularly sensitive regions of the forearm.

An advantageous aspect of the invention provides that the at least one pad is stiffer than the at least one shell element and/or the at least one shell element is stiffer than the at least one strap element. Accordingly, a targeted massage effect can be achieved with the pad, with the shell element giving the elbow brace its shape. The strap element provides a simple way to fasten the shell element to the forearm.

An advantageous aspect of the invention provides that the at least one shell element has at least one, preferably four, in particular point-shaped, second fastening receptacle(s) for receiving at least one cushion. The second fastening receptacles can also be a recess in the honeycomb structure, in particular designed as an aperture or as a blind hole. Accordingly, at least one pad and at least one cushion can be attached to at least one shell element, either alternatively to each other or in combination. The recess for the at least one pad is preferably arranged in the central region and/or the recess for the cushion is preferably arranged in the edge region of the shell element.

An advantageous aspect of the invention provides that the at least one pad and/or the at least one cushion has at least one fastening portion for engaging in the at least one recess and/or in the at least one second fastening receptacle. The at least one fastening portion can be designed as a protruding pin, with the respective fastening portion interacting with the respective recess in a form-fitting manner. The pad and/or the cushion should preferably be clipped into the shell element.

An advantageous aspect of the invention provides that the at least one pad is arranged between the at least one shell element and the at least one cushion. Accordingly, a homogeneous surface can be provided by the elbow brace for application to the forearm.

An advantageous aspect of the invention provides that the elbow brace is designed for arrangement on the forearm of the patient such that at least one pad rests against the extensor carpi radialis brevis muscle and/or the flexore antebrach muscle of the patient. Therefore, the pad can have a particularly good effect on the forearm muscles.

The elbow brace is designed such that the at least one shell element, in particular two shell elements, and the at least one strap element, in particular two strap elements, form a forearm receptacle for receiving the forearm.

The at least one shell element is curved, the bending of the at least one shell element being less than the bending of the ulnar or radial surface of the forearm. Accordingly, the shell element preferably only comes into contact with the forearm in the region of the pad. The depth effect is thus further improved.

An advantageous aspect of the invention provides that the pad, particularly on its outer side, has a first marking indicating the first configuration, in particular a first symbol and/or a first geometry and/or a first color, preferably the letter “T” for the treatment of tennis elbow, and a second marking indicating the second configuration, in particular a second symbol and/or a second geometry and/or a second color, preferably the letter “G” for the treatment of golfer's elbow. The pad is preferably arranged on the shell element in such a way that either the first marking or the second marking is visible on an outer side of the shell element, depending on the configuration. The first marking or the second marking are preferably visible through the recess of the shell element, in particular the recess of the honeycomb structure. Consequently, even when in the wearing state, it is easily visible from the outside in which configuration the pad is set up.

The object of the invention is also achieved by a pad having the features of claim 16. The invention relates to a pad for an elbow brace, the pad comprising at least one rib arranged on an inner side of the pad extending along a rib axis, wherein the pad is asymmetrically designed on the inner side perpendicular to the rib axis. The inner side, in particular the rib, comes into contact with the patient when the pad is in the wearing state

It is advantageous if the pad on the outer side, in particular the fastening portions of the pad, is symmetrical and the at least one rib is asymmetrical or off-center, in particular relative to the fastening portions. Accordingly, the pad can be arranged on the shell element in two pad configurations, wherein the pad as a whole is basically arranged in the same position on the shell element and only the position of the at least one rib perpendicular to the rib axis is changed. Since the radial forearm muscles (“tennis elbow”) are arranged higher than the ulnar forearm muscles (“golfer's elbow”), a single pad can therefore be used to treat either the radial forearm muscles or the ulnar forearm muscles in both configurations.

An advantageous aspect of the invention provides that the pad has at least one, and in particular two, pad nose(s) extending perpendicular to the rib axis. At least one rib is preferably arranged off-center with respect to the two pad noses.

The pad can be arranged on a previously described elbow brace, in particular on the honeycomb structure of the shell element. The rib achieves a particularly good massage effect on the forearm muscles in the arranged state (wearing state) on the elbow brace. The forearm muscles can be guided towards the rib by means of the pad nose.

An advantageous aspect of the invention provides that the pad has two opposing pad noses, and in particular wherein the at least one rib is arranged off-center with respect to the pad noses. This allows for effective and targeted guidance of the forearm muscles towards the rib.

An advantageous aspect of the invention provides that a first nose length of the first pad nose and a second nose length of the second pad nose are designed differently.

An advantageous aspect of the invention provides that the pad is curved towards the inner side. Therefore, the pad can conform particularly well to the forearm.

An advantageous aspect of the invention provides that the pad has at least one fastening portion on an outer side opposite the inner side for connection to a shell element. The pad can preferably be clipped into the shell element.

Accordingly, the pad can be fastened easily to a shell element of the elbow brace.

The pad is preferably made of plastics material. The pad preferably has a Shore hardness in a range between Shore A 5 and 50, in particular between 10 and 30.

The object of the invention is also achieved by an elbow brace for relieving radial and/or ulnar forearm muscles of a patient, comprising: at least one shell element with a honeycomb structure, at least one strap element for fastening the at least one shell element to a forearm of the patient, and at least one pad as previously described, formed integrally with the shell element and/or the honeycomb structure. Due to the one-piece design of the shell element and the pad, the elbow brace can be arranged on the patient particularly easily.

Further advantages, features, and details emerge from the following description, in which various exemplary embodiments of the invention are illustrated with reference to the drawings. The features mentioned in the claims and in the description may in each case be essential to the invention individually or in any desired combination.

### In the drawings

FIG. 1 is a perspective view of an elbow brace;

FIG. 2 is a further perspective view of an elbow brace;

FIG. 3 is a perspective view of a shell element with a pad;

FIG. 4A is a front view of a shell element with a pad in a first orientation;

FIG. 4B is a front view of a shell element with a pad in a second orientation;

FIG. 5 is a view of a pad;

FIGS. 6, 6A are views of a pad;

FIG. 7 is a perspective view of a cushion;

FIG. 8 is a sectional view of a cushion and two shell elements of an elbow brace;

FIG. 9 is a side view of the elbow brace with the pad in the first configuration for the treatment of tennis elbow; and

FIG. 10 is a side view of the elbow brace with the pad in the second configuration for the treatment of golfer's elbow.

The elbow brace 10 is designed in accordance with FIG. 8 for relieving the radial forearm muscles 12 of a patient and the ulnar forearm muscles 14 of a patient by massaging them or repeatedly applying a force.

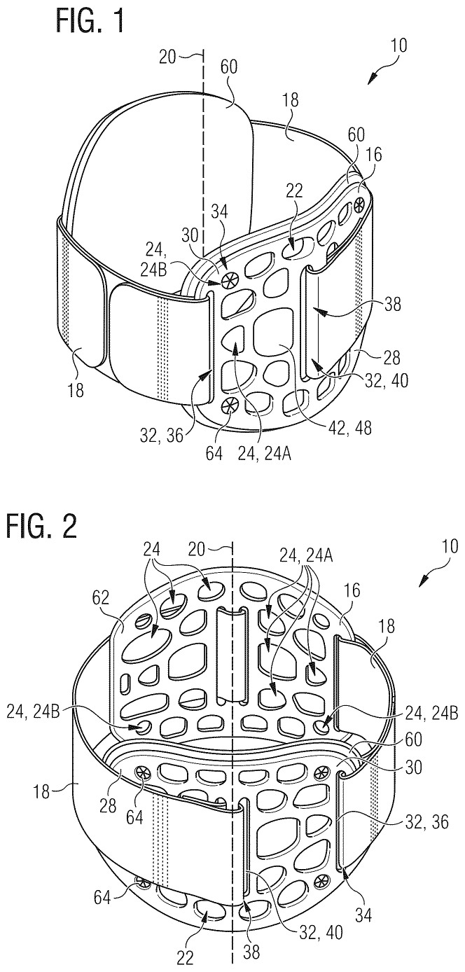

The elbow brace 10 has, in accordance with FIGS. 1, 2 and 8, two shell elements 16 and two strap elements 18 connecting the shell elements 16. The elbow brace 10 is therefore hollow-cylindrical in shape. The elbow brace 10 can be adjusted in its brace width by means of at least one of the two strap elements 18, so that it can be used for different forearm diameters. A hook-and-loop fastener can preferably be used for this purpose. In the applied state, the elbow brace 10 lies around the forearm of the patient, with one of the two shell elements 16 arranged in the region of the radial forearm muscles 12 and another of the two shell elements 16 arranged in the region of the ulnar forearm muscles 14. In the applied state, the forearm extends along a longitudinal axis 20 of the elbow brace 10, wherein the longitudinal axis 20 in the applied state of the elbow brace 10 runs substantially parallel to the muscle fibers of the radial forearm muscles 12 and/or the ulnar forearm muscles 14.

The shell elements 16 are curved towards the longitudinal axis 20 in the applied state, as shown in accordance with FIG. 8. The shell elements 16 preferably each have a honeycomb structure 22 as shown in accordance with FIG. 1 to 4B, which has a plurality of recesses 24 in the form of apertures. The recesses 24 are distributed at least substantially mirror-symmetrically with respect to a mirror axis 26. Larger recesses 24 are provided on a first shell side 28 of the shell element 16 than on a second shell side 30 of the shell element 16 opposite the first shell side 28 perpendicular to the mirror axis 26.

For fastening the strap elements 18, the shell element 16 has first fastening receptacles 32, in particular a first slot 36 in an edge region 34 of the shell element 16 and two second slots 40 in a central region 38 of the shell element 16, wherein the strap elements 18 are each passed through the first slot 36 and the two second slots 40 with a loop. The first slot 36 is preferably arranged on the second shell side 30. Due to the arrangement of a strap element 18 on the central region 38 of the shell element 16, the strap element 18 is guided parallel to the surface of the forearm, which results in a gentler wearing of the elbow brace 10. The first slot 36 and/or the second slots 40 preferably run parallel to the longitudinal axis 20.

The elbow brace 10 further has a pad 42 as shown in accordance with FIG. 1, 3 to 4B and 8, which can be releasably fastened to the shell element 16. The pad 42 is releasably and form-fittingly connected to pad-associated recesses 24A in the honeycomb structure 22. The pad-associated recesses 24A for the pad 42 have a substantially square basic shape with rounded corners. In this case, four pad-associated recesses 24A are arranged, in particular in the middle, to the right, left and rear, on each side, i.e., first shell side 28 and second shell side 30. The pad-associated recesses 24A are preferably arranged in a T-shape. The middle pad-associated recess 24A is preferably larger than the other pad-associated recesses 24A.

The pad 42 has an inner side 44 and an outer side 46. In accordance with FIG. 6 and FIG. 6A, the pad 42 has on its outer side 46 fastening portions 48 with undercuts 50 corresponding to the pad-associated recesses 24A, wherein the fastening portions 48 engage in the pad-associated recesses 24A and the undercuts 50 engage behind the shell element 16 or the honeycomb structure 22. Preferably the middle and rear fastening portions 48 are fully formed and the left and right fastening portions 48 are formed only on the inner side correspondingly to the pad-associated recesses 24A. The fastening portions 48 are designed to be slightly flexible, so that the fastening portions 48 can be easily inserted into the recesses 24. This ensures that the pad 42 is securely held on the shell element 16.

Alternatively, as shown in accordance with FIG. 6A, only two middle and two rear fastening portions 48 may be provided.

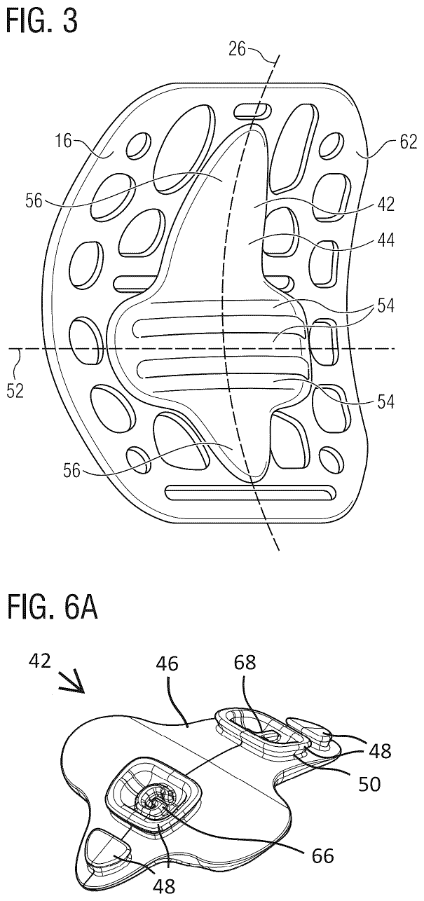

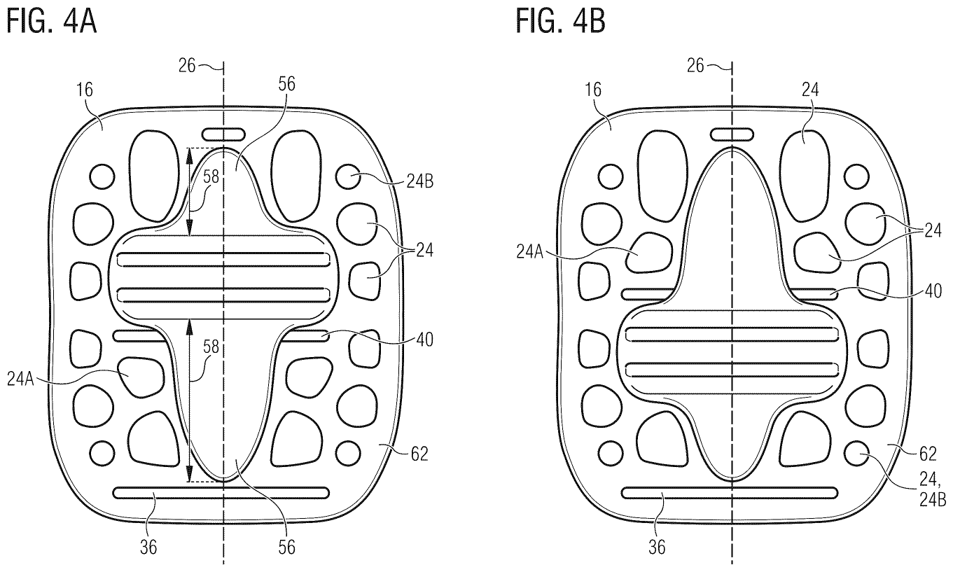

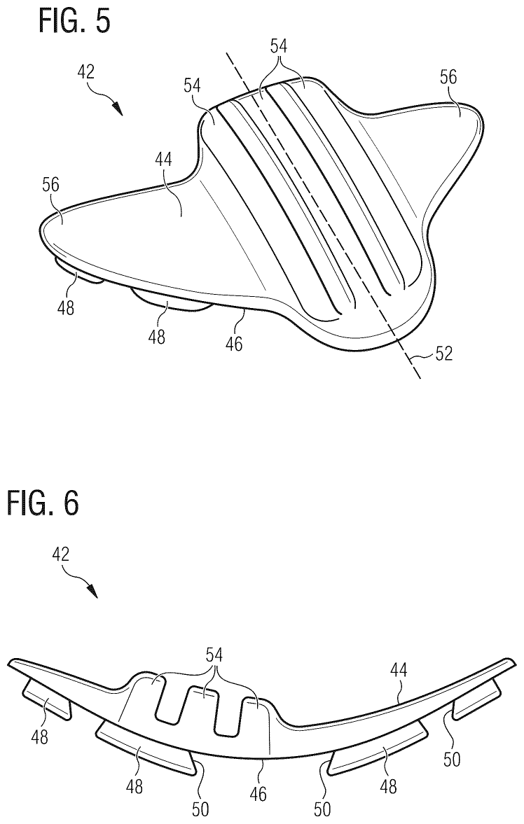

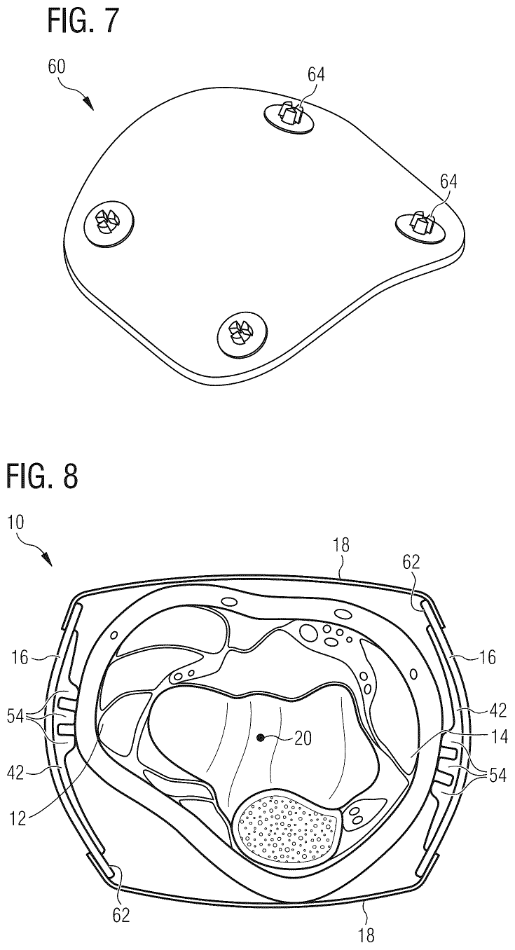

The pad 42 can be arranged in two different orientations on the shell element 16 in accordance with FIGS. 4A and 4B. For this purpose, the fastening portions 48 are preferably designed symmetrically. In accordance with FIG. 3 to 5, the pad 42 has ribs 54 extending on the inner side 44 parallel to a rib axis 52, which preferably runs perpendicular to the mirror axis 26. The ribs 54 serve for a particularly effective and targeted massage effect on the forearm muscles 12. The pad 42 is arranged on the shell element 16 in such a way that the rib axis 52 in the intended wearing state runs at least substantially parallel to muscle fibers of the radial forearm muscles 12 and/or ulnar forearm muscles 14 of the patient. Accordingly, an optimal depth effect of the micromassage of the forearm muscles 12 can be achieved. Due to the different orientations of the pad 42, it can be applied either to the radial forearm muscles 12 or to the ulnar forearm muscles 14 of the patient. For this purpose, the ribs 54 are arranged off-center relative to the fastening portions 48. This is particularly evident from the fact that the pad 42 has two opposing pad noses 56 extending perpendicular to the rib axis 52, which have different nose lengths 58. Accordingly, the inner side 44 of the pad 42 is asymmetrical with respect to the ribs 54 and the outer side 46 of the pad 42 is symmetrical with respect to the fastening portions 48.

The pad noses 56 are preferably designed in such a way that they exert a force on the muscle fibers towards the ribs 54, so that the muscle fibers are at least slightly pushed towards the ribs 54.

The first fastening receptacle 32 extends perpendicular to the mirror axis 26. The first fastening receptacle 32, as shown in accordance with FIG. 2, is preferably arranged between the four pad-associated recesses 24A of the first shell side 28 and the second shell side 30. Accordingly, the pad-associated recesses 24A are also arranged symmetrically to the central region 38.

Along the mirror axis 26, the shell element 16 is longer than the pad 42.

Furthermore, the elbow brace 10 in accordance with FIG. 2 has a cushion 60 or in accordance with FIG. 1 two pads 60 for arrangement on a shell inner side 62 of the shell element 16. The cushion 60 can be arranged directly on the shell element 16, as shown in FIG. 2. Alternatively, a pad 42 can be provided between the shell element 16 and the cushion 60, as shown in FIG. 1. In addition, the honeycomb structure 22 has four, in particular point-shaped, cushion-associated recesses 24B in the edge region 34, into which cushion fastening portions 64 can engage. The cushion fastening portions 64 can also have undercuts 50. The cushion 60 preferably completely covers the shell element 16, so that the skin of the patient is only contacted by the cushion 60.

The shell element 16 is preferably curved towards the shell inner side 62 or to the longitudinal axis 20. The pad 42 is preferably curved towards the inner side 44 or towards the longitudinal axis 20. The cushion 60 is flat and, in the wearing state, is curved towards the longitudinal axis 20.

The elbow brace 10 is shown in FIG. 8 in the wearing state, i.e., in the state arranged on the forearm of the patient. The elbow brace 10 has two identical shell elements 16 as shown in accordance with FIG. 8. For the treatment of the radial forearm muscles 12, the pad 42 is arranged in the first orientation in accordance with FIG. 4A on the shell element 16 and is therefore located higher. For the treatment of the ulnar forearm muscles 14, the pad 42 is arranged in the second orientation in accordance with FIG. 4B on the shell element 16 and is therefore located lower. It is conceivable that two shell elements 16 and one pad 42 are used. It is also conceivable that two shell elements 16, each with a pad 42, are used. The two shell elements 16 are connected to each other on the top and bottom sides by a strap element 18. Accordingly, the elbow brace 10 can be adjusted to the size of the forearm by means of the strap elements 18. Due to the flexibility of the shell elements 16 resulting from the honeycomb structure 22, these nestle against the forearm through the pull of the strap element 18.

In the wearing state, the elbow brace 10 preferably lies against the forearm of the patient in such a way that at least one pad 42 rests against the extensor carpi radialis brevis muscle and/or the flexore antebrach muscle of the patient.

It is also conceivable that the shell elements 16 and the pad 42 and/or the cushion 60 are designed to be non-releasable from each other. The elbow brace 10 can be designed as a single piece or in multiple parts.

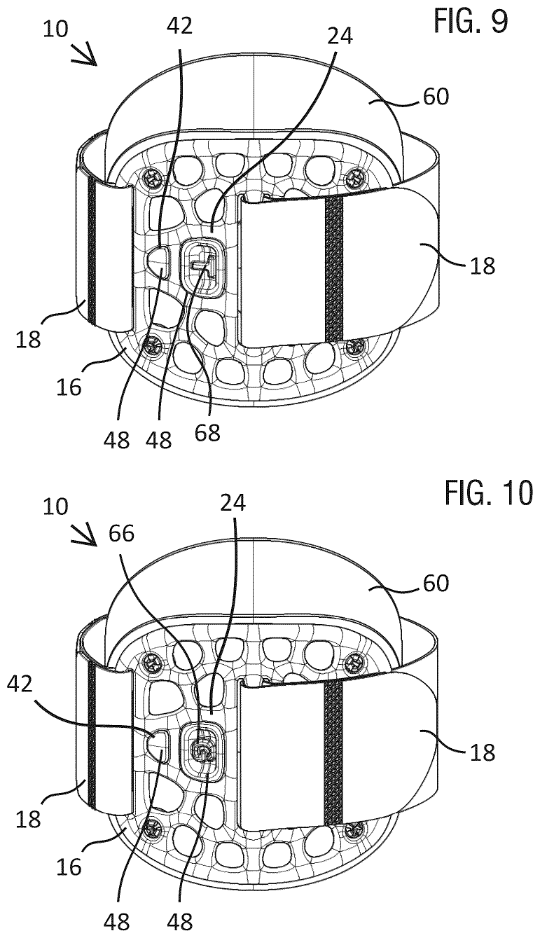

In accordance with FIGS. 9 and 10, the pad 42 has a first marking 66 in the form of a raised letter “G” and a second marking 68 in the form of a raised letter “T” on the outer side 46. The pad 42 can be arranged on the shell element 16 in accordance with FIG. 9 in such a way that in the first configuration or in the first pad orientation only the first marking 66 is visible from the outside through the shell element 16 (“tennis elbow”). The second marking 68 is covered in the second configuration by the strap element 18 and/or the shell element 16. The pad 42 can be arranged on the shell element 16 in such a way that in the second configuration or in the second pad orientation only the second marking 68 is visible from the outside through the shell element 16 (“golfer's elbow”). In the second configuration, the first marking 66 is covered by the strap element 18 and/or the shell element 16. Although the pad 42 is substantially covered by the shell element 16, it is possible to see from the outside in what configuration the pad 42 is set up. Preferably the first marking 66 and/or the second marking 68 is arranged on or in a fastening portion 48. If a first marking 66 and/or a second marking 68 is provided on the pad 42, the fastening portion 48 can be hollow so that the first marking 66 and/or the second marking 68 does not protrude from the fastening portion 48.

1. An elbow brace (10) for relieving radial forearm muscles (12) and/or ulnar forearm muscles (14) of a patient, comprising:
at least one shell element (16) for medial and/or lateral arrangement on the forearm of the patient;
at least one strap element (18) for fastening the at least one shell element (16) to the forearm of the patient;
at least one pad (42) arranged in a defined manner on the at least one shell element (16) with at least one rib (54) extending along a rib axis (52), wherein the at least one pad (42) can be set up in a defined first configuration for the treatment of the radial forearm muscles (12) and in a defined second configuration for the treatment of the ulnar forearm muscles (14).

2. The elbow brace (10) according to claim 1, wherein the at least one shell element (16) and the at least one pad (42) are releasably or non-releasably connected to each other.

3. The elbow brace (10) according to claim 1, wherein the at least one shell element (16) has at least one recess (24) and/or the at least one pad (42) has at least one fastening portion (48) for form-fitting and releasable connection of the at least one shell element (16) and the at least one pad (42).

4. The elbow brace (10) according to claim 1, wherein the at least one pad (42) is asymmetrically formed perpendicular to the rib axis (52), including wherein the at least one rib (54) has a transverse extension and is formed off-center with respect to the transverse extension perpendicular to the rib axis (52).

5. The elbow brace (10) according to claim 1, wherein the at least one pad (42) in the defined first configuration is arranged in a first pad orientation on the at least one shell element (16), wherein the at least one pad (42) in the defined second configuration is arranged in a second pad orientation on the at least one shell element (16), and wherein the first pad orientation and the second pad orientation are inverted relative to each other.

6. The elbow brace (10) according to claim 1, wherein the at least one shell element (16) in the defined first configuration of the at least one pad (42) is arranged in a first shell orientation on the at least one strap element (18) and/or on the forearm of the patient, wherein the at least one shell element (16) in the defined second configuration of the at least one pad (42) is arranged in a second shell orientation on the at least one strap element (18) and/or on the forearm of the patient, and wherein the first shell orientation and the second shell orientation are inverted relative to each other.

7. The elbow brace (10) according to claim 1, wherein the elbow brace (10) comprises a first shell element (16) and a second shell element (16), including wherein first and second shell elements (16) are connected to each other at two opposite ends via two strap elements (18), wherein a first strap element (18) serves to determine the distance between the first and second shell elements (16) and/or the second strap element (18) serves to apply the elbow brace (10), wherein in the defined first configuration the pad (42) for treating the radial forearm muscles (12) can be arranged on the first shell element (16), including in a first pad orientation, and in the defined second configuration for treating the ulnar forearm muscles (14) can be arranged on the second shell element (16) including in a second pad orientation, wherein the first and second shell elements (16) are arranged in the same shell orientation.

8. The elbow brace (10) according to claim 1, wherein the at least one shell element (16) has at least one first recess (24) and at least one second recess (24) provided therein, and wherein the at least one pad (42) is arranged in the defined first configuration at the at least one first recess (24) and in the defined second configuration at the at least one second recess (24).

9. The elbow brace (10) according to claim 1, wherein the at least one shell element (16) has a honeycomb structure (22) has at least one recess (24), including wherein the at least one recess (24) is part of the honeycomb structure (22). (Currently amended) The elbow brace (10) according to claim 1, wherein the at least one shell element (16) is configured to be connected to the at least one strap element (18), and/or the at least one pad (42) is configured to be connected to the shell element (16) so in the rib axis (52) runs at least substantially parallel to muscle fibers of the radial and/or ulnar forearm muscles (12, 14) of the patient.

11. The elbow brace (10) according to claim 1, wherein the elbow brace (10) has two shell elements (16) that are separate from and identical to each other.

12. The elbow brace (10) according to claim 1, wherein the at least one shell element (16) has at least one, first fastening receptacle (32) for the releasable or non-releasable receiving of the at least one strap element (18), including wherein the at least one first fastening receptacle

13. The elbow brace (10) according to claim 12, wherein the first fastening receptacle (32) has a primary fastening receptacle (40) and a secondary fastening receptacle (36), wherein the primary fastening receptacle (40) is arranged in a central region (38) and/or the secondary fastening receptacle (36) is arranged in an edge region (34) of the at least one shell element (16).

14. The elbow brace (10) according to claim 1, any of wherein the at least one shell element (16) has at least one second fastening receptacle (32) for receiving at least one cushion (60), including wherein the at least one second fastening receptacle (32) is point-shaped, and wherein the at least one pad (42) is arranged between the at least one shell element (16) and the at least one pad (60).

15. The elbow brace (10) according to claim 1, wherein the elbow brace (10), including the pad (42), has a first marking (66) characterizing the treatment of the ulnar forearm muscles (14) and a second marking (68) characterizing the treatment of the radial forearm muscles (12), including wherein in the defined first configuration of the at least one pad (42) only has the first marking (66) and in the defined second configuration of the at least one pad (42) only, the second marking (68) is visible.

16. A pad (42) for an elbow brace (10), the pad (42) comprising at least one rib (54) arranged on an inner side (44) of the pad (42) extending along a rib axis (52), wherein the pad (42) is asymmetrically designed on the inner side (44) perpendicular to the rib axis (52).

17. The pad (42) according to claim 16, wherein the pad (42) has two pad noses (56) extending perpendicular to the rib axis (52) and opposite each other, including wherein the at least one rib (54) is arranged off-center with respect to the two pad noses (56).

18. The pad (42) according to claim 17, wherein a first nose length (58) of a first pad nose (56) and a second nose length (58) of a second pad nose (56) are different.

19. The pad (42) according to claim 16, wherein the pad (42) is curved towards the inner side (44).

20. The pad (42) according to claim 16, wherein the pad (42) has at least one fastening portion (48) on an outer side (46) opposite the inner side for connection to a shell element (16).

21. The pad (42) according to claim 16, wherein the pad (42) has an outer side (46) opposite the inner side (44) having a first marking (66) indicating the defined first configuration and a second marking (68) indicating the defined second configuration.
